(19) United States
(12) Patent Application Publication
(10) Pub. No.: US 2025/0299839 A1
(43) Pub. Date: Sep. 25, 2025

(54) SYSTEMS AND METHODS FOR SUPPORTING HEALTH-BASED COMMUNICATIONS

(71) Applicant: Sequelae, Inc., Minneapolis, MN (US)

(72) Inventors: James LaBelle, Encinitas, CA (US); Sara Taylor, Edina, MN (US); John Michael Gray, San Diego, CA (US); Shane Hoversten, Brooklyn Park, MN (US); Mark Briesacher, Park City, UT (US); Steven J. Catani, Athens, GA (US)

(73) Assignee: Sequelae, Inc., Minneapolis, MN (US)

(21) Appl. No.: 19/083,908

(22) Filed: Mar. 19, 2025

Related U.S. Application Data
(60) Provisional application No. 63/568,160, filed on Mar. 21, 2024.

(51) Int. Cl. G16H 80/00; A61B 10/00; G16H 20/00; G16H 50/20

(52) U.S. Cl. CPC G16H 80/00; A61B 10/00; G16H 20/00; G16H 50/20

(57) ABSTRACT

Systems and methods for supporting health-based communications are provided. A digital communication network (DCN) for a plurality of members of a population to interact with each other and the DCN is provided. An invitation is transmitted to an individual member of the plurality of members to test a biological marker related to the adverse health condition and instructions are provided to the individual member to test the biological marker. A test result of the biological marker is received through the DCN and the individual member is enabled to share the test result with other members of the plurality of members through the DCN.

### CROSS REFERENCE TO RELATED APPLICATIONS

This application claims the benefit of U.S. Provisional Application No. 63/568,160, filed on Mar. 21, 2024, which application is incorporated herein by reference in its entirety.

### BACKGROUND

Various embodiments relate generally to health care systems, methods, devices and computer programs and, more specifically, relate to supporting health-based communications through a digital communication network.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Computers have changed the way people interact. Digital networks, which may include the use of social media, allow individuals to interact with others online and connect many people within a community. The online community can be utilized to help build positive behaviors and encourage people to make improvements in their lives through health-based communications.

What is needed is a way to build upon the social media and the access that computers have in order to build healthy lifestyles and encourage members to develop healthy behaviors.

### SUMMARY

Example aspects of the present disclosure include:

A method to manage adverse health conditions in a population according to at least one embodiment of the present disclosure comprises providing a digital communication network (DCN) for a plurality of members of a population to interact with each other and the DCN; transmitting an invitation to an individual member of the plurality of members to test a biological marker related to the adverse health condition; providing instructions to the individual member to test the biological marker; receiving, through the DCN, a test result of the biological marker; and enabling the individual member to share the test result with other members of the plurality of members through the DCN.

Any of the aspects herein, wherein the biological marker is at least one of a chronic systematic inflammation level and an auto-antibody level.

Any of the aspects herein, wherein the biological marker is related to at least one of: inflammation, auto-immune disease, metabolic disease, diabetes, obesity, rheumatoid arthritis (RA), Crohn's disease, Psoriasis, eczema, cardiovascular diseases (CVD), congestive heart failure (CHF), chronic obstructive pulmonary disease (COPD), asthma, depression, anxiety, social isolation, risk of falling, and a prodromal form of the adverse health condition.

Any of the aspects herein, wherein providing instructions to the individual member test the biological marker comprising sending the user a sampling kit to test the biological marker at home.

Any of the aspects herein, wherein the sampling kit is configured to collect at least one of: stool, blood, and saliva.

Any of the aspects herein, wherein the sampling kit comprises at least one of a psychometric instrument, a heart rate monitor, an accelerometer, and a pulse oximeter.

Any of the aspects herein, further comprising prioritizing communications presented to the individual member from other members of the DCN that have levels of the biological marker which are improved over time.

Any of the aspects herein, further comprising prioritizing messages presented to the individual member which are from other members of the DCN engaged in actions aimed at improving respective levels of their biological marker.

Any of the aspects herein, further comprising transmitting an invitation to the individual member to conduct a subsequent test of the biological marker.

Any of the aspects herein, further comprising determining a change in the biological marker based on test result and a subsequent test result from the subsequent test.

Any of the aspects herein, further comprising prioritizing messages presented to the individual member which are from other members of the DCN for which the change in a level of the biological marker is improved over time.

Any of the aspects herein, wherein the invitation to test the biological marker is algorithmically generated.

Any of the aspects herein, wherein the invitation to test the biological marker is transmitted from another member in the DCN.

Any of the aspects herein, further comprising transmitting one or more communications regarding the biological marker created by a bot in the DCN.

A method to manage adverse health conditions in a population according to at least one embodiment of the present disclosure comprises providing a digital communication network (DCN) for a plurality of members of a population to interact with each other and the DCN; transmitting an invitation to an individual member of the plurality of members to conduct a first test and at least a second test of a biological marker related to the adverse health condition; providing instructions to the individual member to conduct the first test and the second test of the biological marker; receiving, through the DCN, a first test result from the first test and a second test result from the second test of the biological marker; and enabling the individual member to share at least one of the first test result, the second test result, and a difference in the first test result and the second test result with other members of the plurality of members through the DCN.

Any of the aspects herein, further comprising transmitting a suggestion of a medical intervention to the individual member based on the difference in the first test result and the second test result.

Any of the aspects herein, wherein each member of the plurality of members conducted the first test and the second test and shared least one of the first test result, the second test result, and a difference in the first test result and the second test result within the DCN, and wherein the method further comprises transmitting a suggestion of a medical intervention to a subset of members based on the difference in the first test result and the second test result, wherein the subset of members have similar differences.

Any of the aspects herein, wherein each member of the plurality of members conducted the first test and the second test and shared least one of the first test result, the second test result, and a difference in the first test result and the second test result within the DCN, and wherein the method further comprises transmitting information about an availability of an intervention paid for by a health care risk holder based on at least partially the difference in the first test result and the second test result.

A method to manage adverse health conditions in a population according to at least one embodiment of the present disclosure comprises providing a digital communication network (DCN) for a plurality of members of a population to interact with each other and the DCN; transmitting an invitation to an individual member of the plurality of members to conduct a first test of a biological marker related to the adverse health condition; providing instructions to the individual member to test the biological marker; receiving, through the DCN, a first test result of the biological marker; enabling the individual member to share the first test result with other members of the plurality of members through the DCN; transmitting an invitation to the individual member to conduct a second test of the biological marker; receiving, through the DCN, a second test result of the biological marker; enabling the individual member to share at least one of the first test result, the second test result, and a difference in the first test result and the second test result with other members of the plurality of members through the DCN; and prioritizing communications presented to the individual member from other members of the DCN that have levels of the biological marker which are improved over time.

Any of the aspects herein, wherein the biological marker is at least one of a chronic systematic inflammation level and an auto-antibody level.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3 is a logic flow diagram that illustrates the operation of a method, in accordance with one or more embodiments.

FIG. 4 is a logic flow diagram that illustrates the operation of a method, in accordance with one or more embodiments.

FIG. 5A shows an example of a computing system, in accordance with one or more embodiments.

FIG. 5B shows an example of a network, in accordance with one or more embodiments.

### DETAILED DESCRIPTION

Various embodiments are directed to managing a member's health and the health system based on a member's progression rather than focus on the member's particular state at the time. Such management may be based in part on creating and implementing changes to a member's behavior. Behavioral changes can be improved by making it a social process instead of an individualistic pursuit.

By way of background, as a social process, value can be placed on those members of a community or population that have experience over expertise. Such value can be used to create an atmosphere or community where members can learn about others who have undergone the same interventions that an individual member is contemplating or in the process of doing. Additionally, the community can be used to help support the individual member, which can be a patient under the care of a physician or simply someone interested in the condition and/or disease.

The community can also be used to encourage lifestyle interventions, which are inherently safe. Such community may use the philosophy that any action now is preferred to a “better” action later and also support the concept that ideas and communication are health care. Conventionally, lifestyle change has been looked at as an individual pursuit, such as plans personalized just for the individual. Further, medicine is typically a one-on-one activity (reinforced by the privacy concepts the system is based on). In the present disclosure, lifestyle change is seen as highly driven by social parameters and the impact on social parameters is critical, as will be discussed below.

Conventional lifestyle applications may tell individuals the “right thing to do,” which could be right, but given the complexity of the lifestyle change, is likely not to occur. Often, if the lifestyle change suggested works, they can make the individual more dependent on things outside the individual's control. On the other hand, communities share experiences, not expertise, which individuals can try and if they work for them, is a success. In some cases, success can range from slowing the progression of adverse conditions to managing a disease, or the overall risk level in a population.

Lifestyle change may be supported by communities that provide support, ideas, and, in the case of these ideas, access to tools to provide objective data to make meaningful lifestyle changes in an individual user or members of a population. For example, in a community where a person is a peer, the actions they take and learn from are their volition and may result in increased agency (or autonomy) or self-efficacy. This not only increases the chance of continuous lifestyle improvement, but improved outcomes throughout the heath system.

Another benefit to various embodiments address an issue where insurers use the lack of an objective measure to block access to care. For example, if a patient has not already tried a weight loss program, then they cannot use a drug to aid them in weight loss or they cannot get bariatric surgery. Rather, by looking at a patient's history, biological markers and/or other factors, patients may be considered for treatment or other care (such as interventions, etc.) rather than sticking to a rigid progression. Patients may be granted access to different treatments in view of characteristics that indicate earlier treatments in the progression may not be well received. The focus is on progression, not state. Thus, when looking at a treatment the absolute value and their variation are not as important as looking at the change in an individual. This change also makes what might look like a noisy marker much more precise.

Further, online communities such as the DCN can be provided so that people can learn about healthy lifestyle practices and work to improve their health between clinical touchpoints, such as office visits. To help incentivize healthy behaviors, individuals can use tools like in-home tests and biosensors that measure how well their health actions are working.

Various embodiments can serve to manage a variety of conditions, such as, CSI. For example, specific inflammatory markers may be of interest, e.g., in diagnosing and approving drugs to treat rheumatoid arthritis (RA). Management of a condition can include maintaining the condition at the present levels, slowing the progression or reversing the condition (if possible). Using the DCN, many members of the population can be assisted which allows the embodiments to be used to manage the health of the population, the health risks of the population and/or reduce health care expense of the population overall.

By way of background, generalized CSI is not specific to any disease and, as such, does not often fit into a single category in a health care system. Such characterization results in CSI not being treated in a risk/benefit positive way with drugs or surgery or with any of the tools the provider and insurer community provide. Instead, it is driver and marker of disease progression in general, even if the particular disease is unknown. While the risk-benefit-ratio of drugs and surgery are not positive it can be improved (and therefore disease progression can be slowed) with lifestyle, social situation, emotional management with little or no risk.

Although CSI effects progression of a disease, progression is not typically measured in health care. Inflammation and its management impact disease progression and, at some point, disease state. If disease progression can be slowed, the disease state can be avoided and the population is less sick. Thus, it is beneficial to provide methods and systems where members of a population can be identified as having increased levels of biological markers that indicate the start of or progression of a disease and providing interventions such as communications from other members experiencing the same increase levels of the biological markers and how those members improved and decreased their levels of the biological markers.

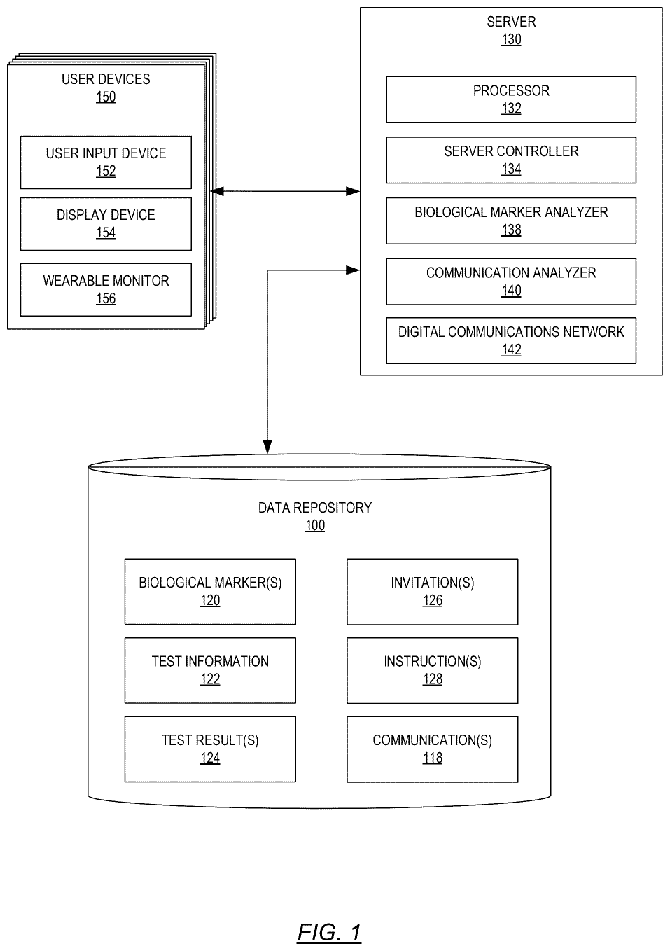

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores biological marker(s) (120). The biological markers (120) are a measurable characteristic of a biological state or condition or what is happening in an organism or a cell. The biological markers (120) are measured and evaluated using a blood, urine, stool, saliva, breath, or soft tissue sample from a user or member of a population or from heart rate meters, accelerometer samples, body temperature as well as other signals derived from wearables. The biological markers (120) can also be derived from psychometric instruments and EMA-derived data. The biological markers (120) can be obtained from testing at home or at a clinic. In instances where the biological markers (120) are obtained from at-home tests, the results may be more precise, which can be used to detect benefits that would otherwise be within the variance of a population-controlled sample.

The biological markers (120) can be indicative or provide information about, for example, a chronic medical condition. The chronic medical condition may be, for example, chronic systemic inflammation (CSI). More specifically, CSI may be measured based on biological markers (120) of activation of a member's immune system. Other biological markers (120) may include auto-antibody levels, cytokines to antibodies, or biomarkers like heart rate variability. Any biological marker (120) may be used and may be specific biological markers (120), general biological markers (120), or may be used as part of an index.

CSI is not specific to any disease, but is rather a driver and marker of disease progression of a disease, such as, for example, Crohn's disease, eczema, psoriasis, etc. Thus, monitoring and treating CSI using the biological markers (120) can lead to positive outcomes for a member of the population. For example, an increasing level of the biological markers (120) may indicate an increase in a member's CSI and the corresponding disease, whereas a decreasing level may indicate a decrease in the member's CSI and severity of the disease. Thus, a member's actions or activities that relate to a reducing level of the biological marker (120) may be beneficial to share with other members of the population with the same or similar biological level. By sharing the member's actions or activities, the other members may be willing to try such actions or activities to reduce their own level of the biological marker (120).

The biological marker (120) may include multiple biological markers (120) obtained from a member at different time periods. For example, the biological marker (120) may include a first biological marker and a second biological marker obtained at different times. The difference between the first biological marker and the second biological marker can be used to determine if a level of the biological marker is increasing or decreasing. It will be appreciated that the biological marker (120) can have any number of biological markers (120), such as one biological marker (120), two biological markers (120), or more than two biological markers (120).

The data repository (100) also stores invitation(s) (126). The invitation (126) may be, for example, an invitation or request to conduct or complete a test for the biological marker (120) to an individual member of the population. In other embodiments, the invitation (126) may be sent to multiple members of the population or the entire population. The invitation (126) may include buttons (e.g., yes/no buttons), and/or a text, and/or media box for the individual member to type in to send feedback such as, for example, more information about the test.

The data repository (100) also stores test information (122). The test information (122) includes information about the test for the biological marker(s) (120) that was presented or listed in the invitation (126) to the individual member. The test information (122) may be in the invitation (126) itself, may be sent upon request from the individual member to the individual member, or may be sent to the individual member upon approval of taking the test from the individual member. The test information (122) may include information, such as the type of test, the duration of the test, whether the test can be conducted at home or at a clinic, information about how or where to obtain an at-home test, and/or information about clinic locations and hours to take the test.

The data repository (100) also stores instruction(s) (128). For at-home tests, the instructions (128) may include instructions for how to obtain the at-home tests and how to administer the at-home tests. For tests taken at a clinic, the instructions (128) may include instructions (128) for how to prepare for the test at the clinic, such as, for example, fasting, dietary restrictions, etc.

The data repository (100) also stores test result(s) (124). The test result(s) (124) may include, for example, one or more level(s) of the biological marker(s) (120) obtained from one or more tests conducted by the individual member. The test results (124) may be received directly from the individual member sending the test results (124) to the DCN or from a clinician.

The data repository (100) also stores communication(s) (118). The communications (118) are communications from members in the DCN (142). The communications may be, for example, text-based, image-based, multimedia communications, audio communications, or any combinations thereof. The communications may be between members, communications in a forum, communications within the DCN (142), or communications generated by the DCN (142) (e.g., communications from a chatbot). The communications (118) can include information about, for example, an individual member's test results (124), a change in the individual member's test results (124), activities or actions taken with respect to the change in the individual member's test results (124), or the like.

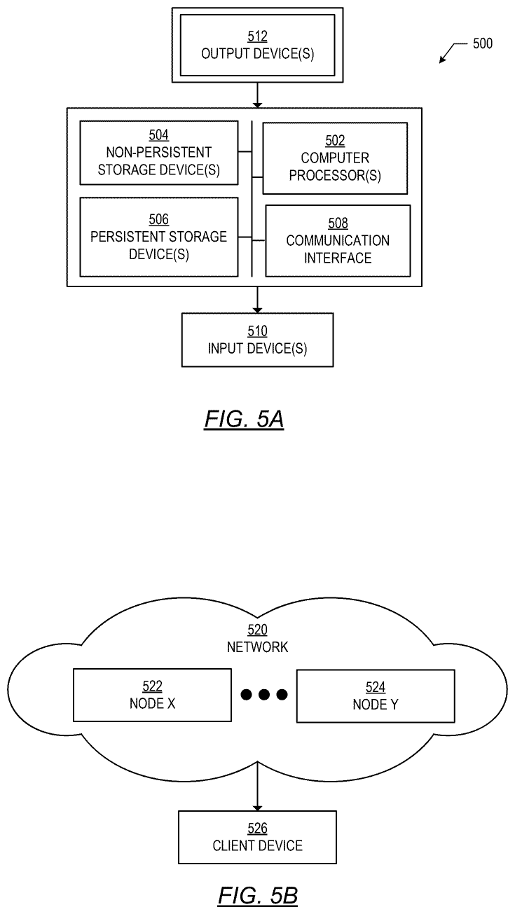

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such a biological marker analyzer (138) or a communication analyzer (140). An example of a computer system and network that may form the server (130) is described with respect to FIG. 5A and FIG. 5B.

The server (130) also includes a computer processor (132). The computer processor (132) is one or more hardware or virtual processors which may execute computer readable program code that defines one or more applications, such as the biological marker analyzer (138) or the communication analyzer (140). An example of the computer processor (132) is described with respect to the computer processor(s) (502) of FIG. 5A.

The server (130) also may include a server controller (134). The server controller (134) is software or application specific hardware which, when executed by the computer processor (526), controls and coordinates operation of the software or application specific hardware described herein.

The server (130) also includes the biological marker analyzer (138). The biological marker analyzer (138) is software or application specific hardware which, when executed by the computer processor (132) provides the level of the biological marker (120). The biological marker analyzer (138) may use a first biological marker, a second biological marker, or any other number of biological markers (120) to determine the level of the biological marker (120). In some embodiments, the biological marker analyzer (138) may determine a difference in the first biological marker and the second biological marker to determine if the level of the biological marker (120) is increasing, decreasing, or has stayed the same.

The server (130) also includes a communication analyzer (140). The communication analyzer (140) is software or application specific hardware which, when executed by the computer processor (132), determines and/or prioritizes communications (118) in the DCN (142) to present a member. For example, the communication analyzer (140) receives, as input, a level of biological marker (120) of the member and outputs the communications (118) that is relevant to the member. The communication analyzer (140) may also prioritize the communications (118) presented to the member. For example, communications (118) that in which other members have levels of the biological marker (120) that have improved over time may be prioritized over communications (118). In another example, communications (118) from members that have engaged in actions aimed at improving respective levels of their biological markers may be prioritized for a member who has an elevated CSI level as indicated by their biological markers (120). In still other examples, communications (126) from members that have engaged in actions to lower their level of CSI-regardless of their level of success-may be prioritized.

The server (130) also includes the digital communications network (DCN) (142). The DCN (142) is a network through which members of a population can interact with each other, or with a system supported by the DCN (142). For example, members can communicate with each other about their chronic medical conditions and activities or interventions that have improved their chronic medical conditions. In at least one embodiment, the DCN (142) can also provide access to activity trackers, health testing, tools to improve a member's health, etc.

In some embodiments, the DCN (142) can receive the biological markers (120) from a member or provide instructions for the member to obtain their biological markers (120) via the test instructions (128) and test information (122). For example, if the member uses an at-home test to obtain their biological markers (120), the DCN (142) can provide instructions (128) for using the at-home test. In other examples, the DCN (142) can provide instructions (128) for visiting a local clinic to obtain their biological markers (120). The DCN (142) can also be used to receive input from the member directly or by a wearable device or monitor (156) that can measure and send information about the member to the DCN (142).

The DCN (142) can also provide means for members of the population to communicate with each other. Members can share their test result (124), differences in multiple test results (124), and/or how they improved their test results (124). The DCN (142) can also be used by providers and individual members to improve, for example, a member's health by improving the member's lifestyle behaviors. For example, members or providers can pass along information and/or tools that enable a member to easily adopt the healthy action that works best for the member.

The DCN (142) also gives access to information and guidance to help members discover healthy actions that work best for them. Members can use the DCN (142) to, for example, find a community of individuals and other members who can support them and vice versa through online or in-person conversations arranged through the DCN (142).

In the DCN (142), the communications (118) may be prioritized. The communications (118) can be prioritized by, for example, the communication analyzer (140). For example, member messages may be ordered to prioritize messages from members who have improved their biological marker (120) levels. In other examples, the communications (118) can be prioritized based on various factors, such as the individual's own biological information, the individuals they follow in the network, etc. These communications (118) between members of the population can be one-to-one, one-to-many, one-to-system, system- to-one, or system-to-many. The system may also feature an AI bot that derives its communications from analysis of communications (118) in the DCN (142) and/or biometrics provided to the DCN (142).

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (500) shown in FIG. 5A) that communicate with the server (130). The user devices (150) may include a wearable monitor (156) and be configured to send stress indicator data (110) to the server (130). In an alternative embodiment, a separate wearable device may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

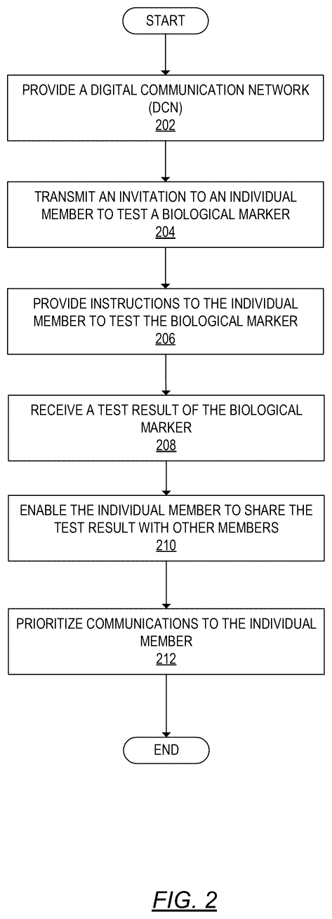

FIG. 2 is a logic flow diagram that illustrates a method, and a result of execution of computer program instructions, in accordance with various embodiments. The method can be used to manage adverse health conditions in a population by enabling members to share results from testing their respective biological markers and activities or methods used to improve their biological markers.

At Block 202, a step of providing a digital communication network (DCN) is provided. The DCN may be the same as or similar to the DCN (142) and provides a network for members of a population to interact with each other or within the DCN (142).

At Block 204, a step of transmitting an invitation to an individual member to test a biological marker is provided. With respect to the methods described herein, the individual member may refer to a single individual member or more than one individual member.

The invitation may be the same as or similar to the invitation (126) and may include an invitation to test for a biological marker such as the biological marker (120). The biological marker may be, for example, CSI or an auto-antibodies. The invitation may include test information such as the test information (122). The invitation may be transmitted to, for example, a user device such as the user device (150) by the DCN. In some embodiments, the invitation to test the biological marker is algorithmically generated. In other embodiments, the invitation to test is transmitted from another member in the DCN.

At Block 206, a step of providing instructions to the individual member to test the biological marker is provided. The instructions may be the same as or similar to the instructions (128). The instructions may include instructions for obtaining an at-home test such as instructions to provide the individual member's address for receiving the at-home test. The at-home test may include a sampling kit to test for the biological marker. In some embodiments, the sampling kit is configured to collect a stool sample, a blood sample, a skin sample, and/or a saliva sample. To obtain such samples, the sampling kit may include a psychometric instrument, a heart rate monitor, an accelerometer, and a pulse oximeter.

In other embodiments, the instructions may include instructions for visiting a clinic to obtain a sample. The instructions may also include information about the clinic, such as opening hours, types of tests available, and types of samples needed.

At Block 208, a step of receiving a test result of the biological marker is provided. The test result may be the same as or similar to the test result (124). The test result may be received by the DCN from the individual member via, for example, a user device such as the user device (150). The test result may be obtained from the individual member using an at-home test or from a provider at a clinic.

In some embodiments, the test result may be received by a direct care provider of the individual member. In such embodiments, the DCN may receive the results from the individual member, then send the results to the direct care provider. In still other embodiments, the individual member may send the at-home test to a testing facility and the test results may be received by the direct care provider and/or the DCN from the test facility.

At Block 210, a step of enabling the individual member to share the test result with other members is provided. The individual member may, for example, receive additional instructions from the DCN to submit a post to the DCN for other members to view. The individual member can share their test results and any reflections or thoughts on the test itself or the test results. For example, if the individual member received a test result that indicated poor health, the individual member may post their test results and seek advice from other members with similar test results.

At Block 212, a step of prioritizing communications to the individual member is provided. Communications such as the communications (118) can be prioritized by, for example, a communication analyzer such as the communication analyzer (140). For example, member messages may be ordered to prioritize messages from members who have improved their biological marker levels as obtained from the test results. In other examples, the communications can be prioritized based on various factors, such as the individual's own biological information, the individuals they follow in the network, etc. Further, the communications can be sent to the individual member's direct care provider. These communications between members of the population can be one-to-one, one-to-many, one-to-system, system-to-one, or system-to-many. The system may also feature an AI bot that derives its communications from analysis of communications in the DCN and/or biometrics provided to the DCN.

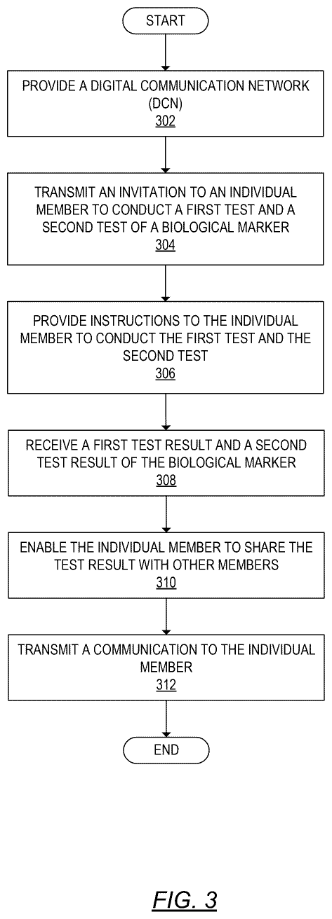

FIG. 3 is a logic flow diagram that illustrates a method, and a result of execution of computer program instructions, in accordance with various embodiments. The method can be used to manage adverse health conditions in a population by enabling members to share a difference in results from testing their respective biological markers multiple times and activities or methods used to improve their biological markers.

At Block 302, a step of providing a digital communication network (DCN) is provided. The Block 302 may be the same as or similar to the Block 202 described above in FIG. 2.

At Block 304, a step of transmitting an invitation to an individual member to conduct a first test and a second test of a biological marker is provided. The Block 304 is the same as or similar to the Block 204 described above in FIG. 2 In the Block 304, the invitation may additionally include a request for the individual member to take the test twice at different times. For example, the second test may be taken hours, days, or weeks after the first test. Such timing can help show a difference in the test results for the individual member and whether their test results are improving, declining, or steady.

It will be appreciated that in other embodiments the Block 304 can includes inviting the individual member (or members) to additional tests, such as a third test, a fourth test, etc.

At Block 306, a step of providing instructions to the individual member to conduct the first test and the second test of the biological marker is provided. The Block 306 is generally the same as or similar to the Block 206 described above in FIG. 2.

At Block 308, a step of receiving a first test result and a second test result of the biological marker is provided. The Block 308 is generally the same as or similar to the Block 208 described above in FIG. 2.

At Block 310, a step of enabling the individual member to share the test result with other members is provided. The Block 310 is generally the same as or similar to the Block 210 described above in FIG. 2. The Block 310 additionally includes that the individual member can share the first test results, the second test results, or the difference between the first test results and the second test results.

At Block 312, a step of transmitting a communication to the individual member is provided. The communication may be the same as or similar to the communication (118). The communication may be transmitted to the individual member may be, for example, created by a bot in the DCN based on the first test result, the second test result, and/or difference in the test result. The communication may also be a suggestion for a medical intervention based on the difference in the first test result and the second test result. For example, the difference in the first test result and the second test result may indicate a medical need for prescription pharmaceuticals and the communication may provide a suggestion for the individual member to visit their physician. In another example where the communication is sent to multiple members with the same or similar differences in the first test result and second test result (or any number of test results), the suggestion may be, for example, to practice meditation daily to reduce stress. In at least one other embodiment, the communication may also include information about an availability of an intervention paid for by a health care risk holder based on at least partially the difference in the first test result and the second test result.

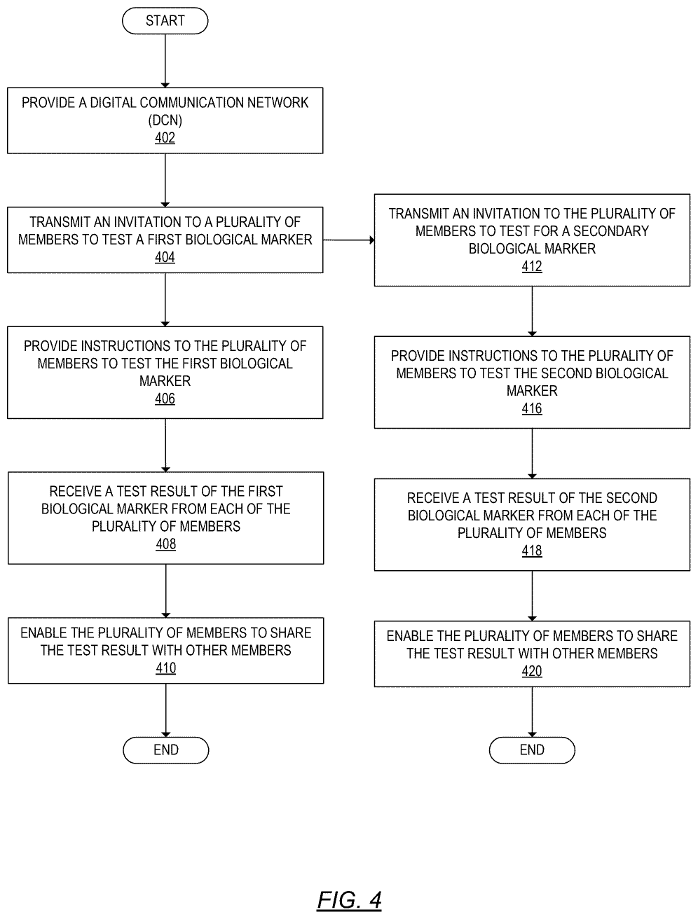

FIG. 4 is a logic flow diagram that illustrates a method, and a result of execution of computer program instructions, in accordance with various embodiments. The method can be used to manage adverse health conditions in a population by enabling members to test for multiple biological markers and sharing results of such testing.

At Block 402, a step of providing a digital communication network (DCN) is provided. The Block 402 may be the same as or similar to the Block 202 described above in FIG. 2.

At Block 404, a step of transmitting an invitation to a plurality of members to test a first biological marker is provided. The Block 404 is the same as or similar to the Block 204 of FIG. 2 described above except that the invitation is sent to a plurality of members.

At Block 406, a step of providing instructions to the plurality of members to test the first biological marker is provided. The Block 406 is generally the same as or similar to the Block 206 of FIG. 2 described above.

At Block 408, a step of receiving a test result of the first biological marker from each member of the plurality of members is provided. The Block 408 is generally the same as or similar to the Block 208 of FIG. 2 described above.

At Block 410, a step of enabling the plurality of members to share the test result with other members is provided. The Block 410 is generally the same as or similar to the Block 210 of FIG. 2 described above.

At Block 412, a step of transmitting an invitation to the plurality of members to test a second biological marker is provided. The Block 412 is generally the same as or similar to the Block 204 of FIG. 2 described above except that the invitation is sent to the plurality of members to make them aware of a second biological marker that they can each test for.

At Block 416, a step of providing instructions to the plurality of members to test the second biological marker is provided. The Block 416 is generally the same as or similar to the Block 206 of FIG. 2 described above.

At Block 418, a step of receiving a test result of the second biological marker from each member of the plurality of members is provided. The Block 418 is generally the same as or similar to the Block 208 of FIG. 2 described above.

At Block 420, a step of enabling the plurality of members to share the test result with other members is provided. The Block 420 is generally the same as or similar to the Block 210 of FIG. 2 described above. The plurality of members may share the test result of the second biological marker with or separately from the test result from the first biological marker.

In at least one example of the methods described above, contract providers typically have looked at the risk of the presence of disease or the risk of a specific disease to evaluate a population and direct care. Chronic systemic inflammation (CSI) is one way to switch the view from the specific disease to the underlying cause of most of the disease and drive to lower health care cost over time. In one embodiment, a method is provided to manage health care risk in a population. The method includes providing the means for members of said population to communicate with each other through a digital communication network (DCN), inviting users of the DCN to measure their level of CSI two or more times, and providing the means for users to share the results of their level of CSI and/or their reflections on their level of systemic inflammation with other members of the DCN.

In such example, the CSI can be measured using a blood or saliva sample. The blood or saliva sample may be taken at home. The systemic inflammation level may be derived from levels in the blood or saliva sample from the measurement of one or more of CRP, IL-2, TNF-alpha, etc. The order of messages shown to a member of the DCN can be derived, in part, from the prioritizing messages from members whose level of CSI is lower over time and/or from members engaged in actions aimed at lowering their level of CSI, regardless of their level of success.

In another example of the methods described above, CSI provides the foundation for one way to manage health care risk in a population. It is the harbinger of other immune system diseases, for example, auto immune disease. Like CSI, measuring the general level of auto-antibodies in a population can be used in a similar way to manage health care costs.

In a further embodiment, a method is provided to manage health care risk in a population. The method includes providing the means for members of said population to communicate with each other through a DCN and inviting/providing the means for users of the social network to measure their level of auto-antibodies two or more times. The method also includes providing users the means to share the results of their level of auto-antibodies and/or their reflections on their level of auto-antibodies with other members of the social network.

The auto-antibody information may be measured using a blood or saliva sample. The blood or saliva sample can be taken at home. Auto-antibody level is derived from levels in the blood or saliva sample from the measurement of one or more enzymes, such as, glutamic acid decarboxylase (GAD). The order of messages shown to a member of the social network may be derived, in part, from prioritizing messages from members whose level of auto-antibodies is lower over time.

In still another example of the methods described above, another embodiment combines CSI with another related measurement in order to provide a method to manage health care risk in a population. The method includes providing the means for members of said population to communicate with each other through a DCN. The method includes inviting and providing the means for users of the digital communications network to measure their level of CSI two or more times. Members of the population are then made aware of a test for a secondary related generalize health marker and invited to measure their level of the secondary related generalize health marker (including providing the means for users of the social network to test that marker).

The order of messages shown to a member of the social network may be derived, in part, from the prioritizing messages from members who have used the secondary related generalize health marker. The secondary marker information may be measured using a blood or saliva sample or a wearable device. The blood or saliva sample can be taken at home. As one non-limiting example, the secondary marker level is related to auto-antibodies. The secondary marker may be related to the member's epigenome. In some embodiments, the secondary marker is related to the member's stress, anxiety, or depression. The secondary related generalized health marker can also be related to the members activity. For example, the secondary marker can be related to the member's social interaction.

One or more embodiments may be implemented on a computing system specifically designed to achieve an improved technological result. When implemented on a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 5A, the computing system (500) may include one or more computer processor(s) (502), non-persistent storage device(s) (504), persistent storage device(s) (506), a communication interface (508) (e.g., Bluetooth interface, infrared interface, network interface, optical interface, etc.), and numerous other elements and functionalities that implement the features and elements of the disclosure. The computer processor(s) (502) may be an integrated circuit for processing instructions. The computer processor(s) (502) may be one or more cores, or micro-cores, of a processor. The computer processor(s) (502) includes one or more processors. The computer processor(s) (502) may include a central processing unit (CPU), a graphics processing unit (GPU), a tensor processing unit (TPU), combinations thereof, etc.

The input device(s) (510) may include a touchscreen, keyboard, mouse, microphone, touchpad, electronic pen, or any other type of input device. The input device(s) (510) may receive inputs from a user that are responsive to data and messages presented by the output device(s) (512). The inputs may include text input, audio input, video input, etc., which may be processed and transmitted by the computing system (500) in accordance with one or more embodiments. The communication interface (508) may include an integrated circuit for connecting the computing system (500) to a network (not shown) (e.g., a local area network (LAN), a wide area network (WAN), such as the Internet, mobile network, or any other type of network) or to another device, such as another computing device, and combinations thereof.

Further, the output device(s) (512) may include a display device, a printer, external storage, or any other output device. One or more of the output device(s) (512) may be the same or different from the input device(s) (510). The input device(s) (510) and output device(s) (512) may be locally or remotely connected to the computer processor(s) (502). Many different types of computing systems exist, and the aforementioned input device(s) (510) and output device(s) (512) may take other forms. The output device(s) (512) may display data and messages that are transmitted and received by the computing system (500). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium, such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (502), is configured to perform one or more embodiments, which may include transmitting, receiving, presenting, and displaying data and messages described in the other figures of the disclosure.

The computing system (500) in FIG. 5A may be connected to, or be a part of, a network. For example, as shown in FIG. 5B, the network (520) may include multiple nodes (e.g., node X (522) and node Y (524), as well as extant intervening nodes between node X (522) and node Y (524)). Each node may correspond to a computing system, such as the computing system shown in FIG. 5A, or a group of nodes combined may correspond to the computing system shown in FIG. 5A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (500) may be located at a remote location and connected to the other elements over a network.

The nodes (e.g., node X (522) and node Y (524)) in the network (520) may be configured to provide services for a client device (526). The services may include receiving requests and transmitting responses to the client device (526). For example, the nodes may be part of a cloud computing system. The client device (526) may be a computing system, such as the computing system shown in FIG. 5A. Further, the client device (526) may include or perform all or a portion of one or more embodiments.

The computing system of FIG. 5A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method to manage adverse health conditions in a population, the method comprising:
providing a digital communication network (DCN) for a plurality of members of a population to interact with each other and the DCN;
transmitting an invitation to an individual member of the plurality of members to test a biological marker related to the adverse health condition;
providing instructions to the individual member to test the biological marker;
receiving, through the DCN, a test result of the biological marker; and
enabling the individual member to share the test result with other members of the plurality of members through the DCN.

2. The method of claim 1, wherein the biological marker is at least one of a chronic systematic inflammation level and an auto-antibody level.

3. The method of claim 1, wherein the biological marker is related to at least one of: inflammation, auto-immune disease, metabolic disease, diabetes, obesity, rheumatoid arthritis (RA), Crohn's disease, Psoriasis, eczema, cardiovascular diseases (CVD), congestive heart failure (CHF), chronic obstructive pulmonary disease (COPD), asthma, depression, anxiety, social isolation, risk of falling, and a prodromal form of the adverse health condition.

4. The method of claim 1, wherein providing instructions to the individual member test the biological marker comprising sending the user a sampling kit to test the biological marker at home.

5. The method of claim 4, wherein the sampling kit is configured to collect at least one of: stool, blood, and saliva.

6. The method of claim 4, wherein the sampling kit comprises at least one of a psychometric instrument, a heart rate monitor, an accelerometer, and a pulse oximeter.

7. The method of claim 1, further comprising prioritizing communications presented to the individual member from other members of the DCN that have levels of the biological marker which are improved over time.

8. The method of claim 1, further comprising prioritizing messages presented to the individual member which are from other members of the DCN engaged in actions aimed at improving respective levels of their biological marker.

9. The method of claim 1, further comprising transmitting an invitation to the individual member to conduct a subsequent test of the biological marker.

10. The method of claim 9, further comprising determining a change in the biological marker based on test result and a subsequent test result from the subsequent test.

11. The method of claim 10, further comprising prioritizing messages presented to the individual member which are from other members of the DCN for which the change in a level of the biological marker is improved over time.

12. The method of claim 1, wherein the invitation to test the biological marker is algorithmically generated.

13. The method of claim 1, wherein the invitation to test the biological marker is transmitted from another member in the DCN.

14. The method of claim 1, further comprising transmitting one or more communications regarding the biological marker created by a bot in the DCN.

15. A method to manage adverse health conditions in a population, the method comprising:
providing a digital communication network (DCN) for a plurality of members of a population to interact with each other and the DCN;
transmitting an invitation to an individual member of the plurality of members to conduct a first test and at least a second test of a biological marker related to the adverse health condition;
providing instructions to the individual member to conduct the first test and the second test of the biological marker;
receiving, through the DCN, a first test result from the first test and a second test result from the second test of the biological marker; and
enabling the individual member to share at least one of the first test result, the second test result, and a difference in the first test result and the second test result with other members of the plurality of members through the DCN.

16. The method of claim 15, further comprising transmitting a suggestion of a medical intervention to the individual member based on the difference in the first test result and the second test result.

17. The method of claim 15, wherein each member of the plurality of members conducted the first test and the second test and shared least one of the first test result, the second test result, and a difference in the first test result and the second test result within the DCN, and wherein the method further comprises transmitting a suggestion of a medical intervention to a subset of members based on the difference in the first test result and the second test result, wherein the subset of members have similar differences.

18. The method of claim 15, wherein each member of the plurality of members conducted the first test and the second test and shared least one of the first test result, the second test result, and a difference in the first test result and the second test result within the DCN, and wherein the method further comprises transmitting information about an availability of an intervention paid for by a health care risk holder based on at least partially the difference in the first test result and the second test result.

19. A method to manage adverse health conditions in a population, the method comprising:
providing a digital communication network (DCN) for a plurality of members of a population to interact with each other and the DCN;
transmitting an invitation to an individual member of the plurality of members to conduct a first test of a biological marker related to the adverse health condition;
providing instructions to the individual member to test the biological marker;
receiving, through the DCN, a first test result of the biological marker;
enabling the individual member to share the first test result with other members of the plurality of members through the DCN;
transmitting an invitation to the individual member to conduct a second test of the biological marker;
receiving, through the DCN, a second test result of the biological marker;
enabling the individual member to share at least one of the first test result, the second test result, and a difference in the first test result and the second test result with other members of the plurality of members through the DCN; and
prioritizing communications presented to the individual member from other members of the DCN that have levels of the biological marker which are improved over time.

20. The method of claim 19, wherein the biological marker is at least one of a chronic systematic inflammation level and an auto-antibody level.
